(19) United States
(12) Patent Application Publication
(10) Pub. No.: US 2025/0182273 A1
(43) Pub. Date: Jun. 5, 2025

(54) IDENTIFICATION AND CHARACTERIZATION OF PANCREATIC CYSTIC LESIONS

(71) Applicant: Bayer Aktiengesellschaft, Leverkusen (DE)

(72) Inventor: Matthias LENGA, Kiel (DE)

(73) Assignee: Bayer Aktiengesellschaft, Leverkusen (DE)

(21) Appl. No.: 18/873,638

(22) PCT Filed: Jun. 16, 2023
(86) PCT No.: PCT/EP2023/066194

§ 371 Date: Dec. 10, 2024

(30) Foreign Application Priority Data
Jun. 19, 2022 (EP) 22179768.1

(51) Int. Cl. G06T 7/00; A61B 5/00; A61B 5/055; A61B 6/46; A61B 6/50; G06V 10/26; G06V 10/764

(52) U.S. Cl. CPC G06T 7/0012; A61B 5/055; A61B 5/425; A61B 6/463; A61B 6/50; G06V 10/26; G06V 10/764; G06T 2207/10116; G06T 2207/30096; G06T 2207/30172; G06V 2201/031

(57) ABSTRACT

The present invention relates to the technical field of the identification and characterization of pancreatic cystic lesions. The present invention relates to a computer-implemented method, a device and a computer-readable storage medium for the identification and characterization of pancreatic cystic lesions.

### CROSS-REFERENCE TO RELATED APPLICATIONS

This application is a U.S. national stage application under 35 U.S.C. § 371 of International Application No. PCT/EP2023/066194, filed internationally on Jun. 16, 2023, which claims benefit of European Application No. 22179768.1, filed Jun. 19, 2022.

### FIELD OF THE INVENTION

The present invention is concerned with the technical field of identification and characterization of cystic lesions of the pancreas. Subjects of the present invention are a computer-implemented method, a device and a computer-readable storage medium for identification and characterization of cystic lesions of the pancreas.

### BACKGROUND

The pancreas is a glandular organ located in the abdomen between the liver, spleen, stomach and duodenum.

The pancreas is an exocrine and an endocrine gland at the same time; as an exocrine gland, it produces digestive enzymes, and as an endocrine gland, it produces hormones (e.g. insulin and glucagon).

The pancreatic juice formed by the pancreas is a water-clear secretion containing enzymes for the digestion of food. The pancreatic juice initially accumulates in small side ducts, which then, similar to a drain system, open into the main pancreatic duct. The main pancreatic duct opens into the duodenum at the so-called papilla.

In the pancreas, cysts can occur. Compared to other organs, cysts in the pancreas are rather rare. They often do not cause any complaints and are frequently discovered by chance. The prevalence in the population is believed to be approx. 2.5%.

There are more than 15 different cyst types in the pancreas and most of these cysts are harmless. However, some cysts in the pancreas can be precursors of a malignant tumor and deteriorate into pancreatic cancer.

Cystic lesions of the pancreas are increasingly detected thanks to the widespread use of high-resolution abdominal imaging methods. Abdominal sonography already allows detection of many cystic changes. The diagnostic method is complemented by modern cross-sectional imaging methods such as computed tomography (CT) and magnetic resonance imaging.

### BRIEF SUMMARY

It is an object of the present invention to assist radiologists in the identification and characterization of cystic lesions of the pancreas.

This object is achieved by the subjects of the independent claims. Preferred embodiments can be found in the dependent claims, the present description and the drawings.

The present invention provides in a first aspect a computer-implemented method comprising the steps of:
receiving at least one radiological image, the at least one radiological image showing a pancreas of an examination object,
segmenting the pancreas from the at least one radiological image,
segmenting parts of the pancreas from the at least one radiological image,
identifying one or more cysts in the identified parts of the pancreas,
classifying the one or more cysts,
determining one or more measurement values for the one or more cysts,
outputting at least one representation of the pancreas, the one or more cysts having been labeled in the at least one representation.

The present invention further provides a device comprising a processor and a working memory in which a computer program can be loaded, the computer program causing the processor to execute the following steps:
loading at least one radiological image into the working memory, the at least one radiological image showing a pancreas of an examination object,
segmenting the pancreas from the at least one radiological image,
segmenting parts of the pancreas in the at least one radiological image,
identifying one or more cysts in the identified parts of the pancreas,
classifying the one or more cysts,
determining one or more measurement values for the one or more cysts,
outputting at least one representation of the pancreas, the one or more cysts having been labeled in the at least one representation.

The present invention further provides a computer-readable storage medium comprising commands which, upon execution by a computer, cause said computer to execute the following steps:
loading at least one radiological image into the working memory, the at least one radiological image showing a pancreas of an examination object,
segmenting the pancreas from the at least one radiological image,
segmenting parts of the pancreas from the at least one radiological image,
identifying one or more cysts in the identified parts of the pancreas,
classifying the one or more cysts,
determining one or more measurement values for the one or more cysts,
outputting at least one representation of the pancreas, the one or more cysts having been labeled in the at least one representation.

### BRIEF DESCRIPTION OF THE DRAWINGS

FIG. 1(a) shows by way of example and in schematic form one embodiment of the method according to the invention for identifying and characterizing cystic lesions of the pancreas.

FIG. 1(b) shows by way of example and in schematic form one embodiment of the method according to the invention for identifying and characterizing cystic lesions of the pancreas.

FIG. 2(a) shows a schematic illustration of the pancreas (A) and the main pancreatic duct (B).

FIG. 2(b) shows a schematic illustration of the pancreas (A), segmented on the basis of the at least one radiological image into four segments (A1, A2, A3, A4), and the segmented main pancreatic duct (B) for determination of the centerline (C).

FIG. 2(c) shows a schematic illustration of the pancreas (A), segmented on the basis of the at least one radiological image into four segments (A1, A2, A3, A4), and a cystic lesion (D) in segment A1 and a further cyst (E) along the segmented main pancreatic duct (B) in segment A3.

### DETAILED DESCRIPTION

The invention will be more particularly elucidated below without distinguishing between the subjects of the invention (method, device, computer program, computer-readable storage medium). Instead, the elucidations that follow are intended to apply analogously to all the subjects of the invention, irrespective of the context (method, device, computer program, computer-readable storage medium) in which they occur.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

In certain places the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say applicable to other embodiments too and not limited to the embodiments shown.

The present invention provides means which assist a radiologist in the identification and characterization of cystic lesions of the pancreas.

A lesion refers to any damage, injury or disorder in an anatomical structure or physiological function. In medical imaging (radiology), areas having altered signal behavior are collectively referred to as lesions, especially because the origin of a lesion (inflammation, parasite, tumor, injury, deterioration and/or others) is often not yet known when a radiological image showing the lesion is generated.

A cyst is a cavity in the tissue of a body that is filled with a fluid in many cases.

Preferably, one or more of the following cystic lesions are identified and characterized: serous cystic neoplasm (SCN), mucinous cystic neoplasm (MCN), intraductal papillary mucinous neoplasm (IPMN), dysontogenetic cysts, pseudocysts.

The identification and characterization of cystic lesions of the pancreas is done on the basis of one or more radiological images.

Thus, in a first step, at least one radiological image is received, the at least one radiological image showing the pancreas of an examination object. The examination object is a vertebrate, preferably a mammal, particularly preferably a human.

The at least one radiological image is preferably at least one computed tomography image (CT image), at least one magnetic resonance image (MRI image) and/or at least one ultrasound image.

The at least one radiological image may be a contrast agent-free image or it may be one or more radiological images before and/or after the administration of one or more contrast agents.

“Contrast agents” are substances or mixtures of substances that improve the depiction of structures and functions of the body in radiological imaging methods. Examples of contrast agents can be found in the literature (see for example A. S. L. Jascinth et al.: Contrast Agents in computed tomography: A Review, Journal of Applied Dental and Medical Sciences, 2016, Vol. 2, Issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nough et al.: Radiographic and magnetic resonances contrast agents: Essentials and tips for safe practices, World J Radiol. 2017 Sep. 28; 9(9):339-349; L. C. Abonyi et al.: Intravascular Contrast Media in Radiography: Historical Development & Review of Risk Factors for Adverse Reactions, South American Journal of Clinical Research, 2016, Vol. 3, Issue 1, 1-10; ACR Manual on Contrast Media, 2020, ISBN: 978-1-55903-012-0; A. Ignee et al.: Ultrasound contrast agents, Endosc Ultrasound. 2016 November-Dec; 5 (6): 355-362).

The at least one radiological image shows the pancreas of the examination object. The at least one radiological image may be a two-dimensional representation of a layer through the pancreas or comprise such a representation. Preferably, the at least one radiological image is a three-dimensional representation of an examination region of the examination object that comprises the pancreas, or a stack of two-dimensional representations of layers within the examination region that form a three-dimensional representation when stacked one on top of the other.

The radiological images are usually in digital form. The term “digital” means that the representations can be processed by a machine, generally a computer system. “Processing” is understood to mean the known methods for electronic data processing (EDP). An example of a customary format for a radiological image is the DICOM format (DICOM: Digital Imaging and Communications in Medicine)—an open standard for storing and exchanging information in medical image-data management.

In a digital radiological image, image contents are usually represented by whole numbers and stored. In most cases, the images are two- or three-dimensional images, which can be binary coded and optionally compressed. The digital radiological image can be a raster graphic, in which the image information is stored in a uniform raster grid. Raster graphics consist of a raster arrangement of so-called picture elements (pixels) in the case of two-dimensional representations or volume elements (voxels) in the case of three-dimensional representations, each of which is assigned a color or a gray level. The main features of a 2D raster graphic are therefore the image size (width and height and optionally depth measured in pixels/voxels, also informally called image resolution) and the color depth. Each picture element/volume element is usually assigned a color or a gray level. The color coding used is defined, inter alia, in terms of the color space and the color depth. The simplest case is a binary image, in which a picture element/volume element stores a black-and-white value. In the case of an image, the color of which is defined in terms of the so-called RGB color space (RGB stands for the primary colors red, green and blue), each picture element/volume element consists of three color values, one color value for the color red, one color value for the color green and one color value for the color blue. The color of a picture element/volume element arises from the superimposition (additive mixing) of the three color values. The individual color value is discretized e.g. into 256 distinguishable levels, which are called tonal values and usually range from 0 to 255. The color nuance “0” of each color channel is usually the darkest. If all three channels have the tonal value 0, the corresponding picture element/volume element appears black; if all three channels have the tonal value 255, the corresponding picture element/volume element appears white. When carrying out the present invention, digital radiological images are subjected to certain operations. The operations affect predominantly the picture elements/volume elements and/or the tonal values of the individual picture elements/volume elements. There are a multiplicity of possible digital image formats, color codings and gray level codings. For simplicity, it is assumed in this description that the present radiological images are raster graphics having a specific number of picture elements/volume elements, each of which has a defined color or gray level. However, this assumption ought not in any way be understood as limiting. It is clear to an expert in image processing how the teaching of this description can be applied to radiological images available in other image formats.

The at least one radiological image of the pancreas may be received from a CT scanner, an MRI scanner and/or an ultrasound device and/or be read from one or more data memories.

The identification and/or characterization of cystic lesions of the pancreas may comprise a number of processes that can build upon each other. These processes will be more particularly elucidated below. Each process usually serves a purpose. To achieve a purpose, there may be multiple processes. Such alternative processes will also be elucidated below. Preferably, the present invention comprises one or more of the following processes: segmentation of the pancreas, segmentation and/or identification of the main pancreatic duct, identification and/or characterization of dilatations in the main pancreatic duct, identification and/or characterization and/or localization of lesions in the pancreas, morphometric examination of the pancreas, visualization of the pancreas.

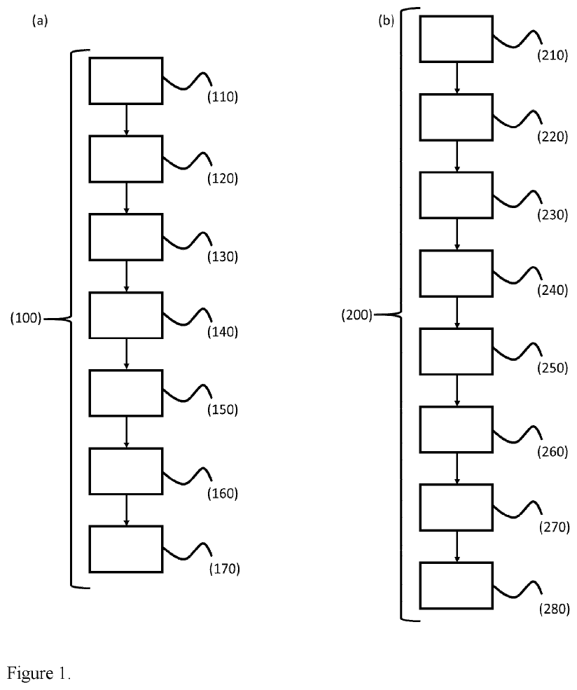

FIG. 1(a) shows by way of example and in schematic form one embodiment of the method according to the invention for identifying and characterizing cystic lesions of the pancreas. Said method (100) comprises the steps of:
(110) receiving at least one radiological image, the at least one radiological image showing a pancreas of an examination object,
(120) segmenting the pancreas from the at least one radiological image,
(130) segmenting parts of the pancreas from the at least one radiological image,
(140) identifying one or more cysts in the identified parts of the pancreas,
(150) classifying the one or more cysts,
(160) determining one or more measurement values for the one or more cysts,
(170) outputting at least one representation of the pancreas, the one or more cysts having been labeled in the at least one representation.

FIG. 1(b) shows by way of example and in schematic form one embodiment of the method according to the invention for identifying and characterizing cystic lesions of the pancreas. Said method (200) comprises the steps of:
(210) receiving at least one radiological image, the at least one radiological image showing a pancreas of an examination object,
(220) segmenting the pancreas from the at least one radiological image,
(230) segmenting parts of the pancreas from the at least one radiological image,
(240) identifying and segmenting the main pancreatic duct
(250) determining the centerline in the main pancreatic duct
(250) identifying one or more cysts in the identified parts of the pancreas or in the main pancreatic duct,
(260) classifying the one or more cysts,
(270) determining one or more measurement values for the one or more cysts,
(280) outputting at least one representation of the pancreas, the one or more cysts having been labeled in the at least one representation.

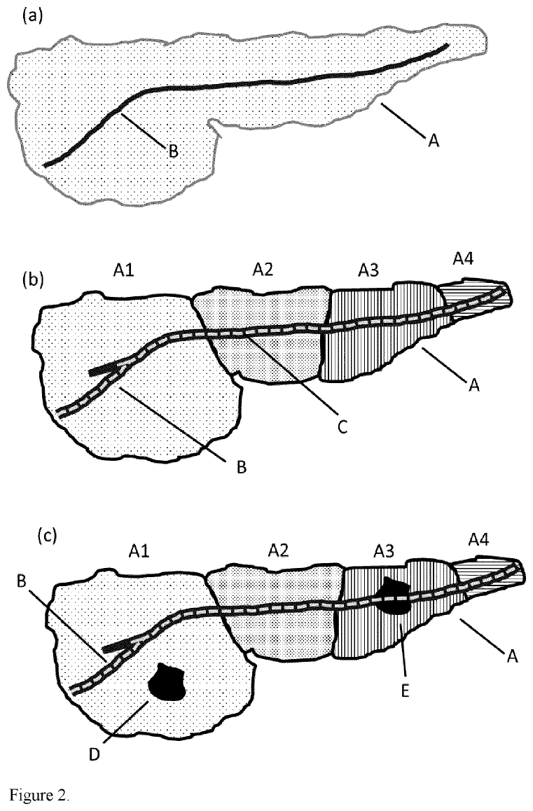

FIG. 2(a) shows a schematic illustration of the pancreas (A) and the main pancreatic duct (B).

FIG. 2(b) shows a schematic illustration of the pancreas (A), segmented on the basis of the at least one radiological image into four segments (A1, A2, A3, A4), and the segmented main pancreatic duct (B) for determination of the centerline (C).

FIG. 2(c) shows a schematic illustration of the pancreas (A), segmented on the basis of the at least one radiological image into four segments (A1, A2, A3, A4), and a cystic lesion (D) in segment A1 and a further cyst (E) along the segmented main pancreatic duct (B) in segment A3.

### Segmentation of the Pancreas

The term “segmentation” is understood to mean the process of dividing a radiological image into multiple segments, which are also referred to as image segments, image regions or image objects. Segmentation is generally used to locate objects and boundaries (lines, curves, etc.) in radiological images. From a segmented radiological image, the objects located can be separated from the background, visually highlighted (e.g. colored), measured, counted, or quantified in some other way.

In segmentation, each picture element/volume element of a radiological image is assigned a label, such that picture elements/volume elements having the same label share certain features.

In the present case, what are identified and labeled in the at least one radiological image are those picture elements/volume elements that represent the pancreas; all other picture elements/volume elements accordingly represent other parts of the examination object.

In a preferred embodiment, the segmentation is done with the aid of a machine learning model which has been trained on the basis of training data to assign each picture element/volume element of a radiological image to one of at least two classes, at least one class representing picture elements/volume elements showing part of the pancreas.

A “machine learning model” can be understood as a computer-implemented data processing architecture. The model can receive input data and supply output data on the basis of these input data and model parameters. The model can learn a relationship between the input data and the output data by means of training. During training, the model parameters can be adapted to supply a desired output for a specific input.

During the training of such a model, the model is presented with training data from which it can learn. The trained machine learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum.

During training, a loss function can be used to assess the prediction quality of the model. The loss function can be chosen such that it rewards a desired relationship between output data and target data and/or punishes an undesired relationship between output data and target data. Such a relationship can be e.g. a similarity, a dissimilarity or some other relationship.

A loss function can be used to calculate a loss value for a given pair of output data and target data. The goal of the training process can consist in altering (adapting) the parameters of the machine learning model so that the loss value for all pairs of the training data set is reduced to a (defined) minimum.

A loss function can quantify e.g. the difference between the output data of the model for specific input data and the target data. For example, if the output data and the target data are numbers, the loss function can be the absolute difference between these numbers. In this case, a high absolute value of the loss function can mean that one or more model parameters must be changed to a great extent.

In the case of output data in the form of vectors, for example, difference metrics between vectors such as the mean squared error, a cosine distance, a norm of the difference vector such as a Euclidean distance, a Chebyshev distance, an Lp norm of a difference vector, a weighted norm or any other type of difference metric of two vectors can be chosen as the loss function.

In the case of higher-dimensional outputs, such as two-dimensional, three-dimensional or higher-dimensional outputs, an element-by-element difference metric can for example be used. Alternatively or additionally, the output data can be transformed before the calculation of a loss value, e.g. into a one-dimensional vector.

In the present case, a machine learning model may be trained to accept as input data a radiological image showing a pancreas and to generate output data at least partly on the basis of the input data and model parameters. The output data may be a radiological image in which each picture element/volume element representing part of the pancreas is labeled. The label may be for example a numeral assigned to each picture element/volume element. The numeral may indicate whether a picture element/volume element represents the pancreas or represents a different part of the examination object. For example, the numeral 1 may indicate that a picture element/volume element represents part of the pancreas and the numeral 0 may indicate that a picture element/volume element does not represent part of the pancreas. It is also possible that a numeral indicates a probability of a picture element/volume element representing part of the pancreas.

The machine learning model may be trained on the basis of training data. The training data may comprise unsegmented and segmented radiological images of the pancreas for a multiplicity of examination objects. For each examination object, at least one unsegmented radiological image and at least one segmented radiological image are usually present. The unsegmented radiological image is fed to the machine learning model as input data. The at least one segmented radiological image serves as target data (also called ground truth) and can be compared with the output data generated by the machine learning model. The segmented radiological images may have been generated by an expert (e.g. a radiologist) on a computer on the basis of the unsegmented radiological images. The expert may for example display the unsegmented radiological images on a screen with the aid of an image editing program and label regions in the unsegmented radiological images that represent the pancreas. The labeled radiological images form the segmented radiological images.

The output data can be compared with the segmented radiological images and the differences between the output data and the segmented radiological images may be quantified with the aid of a loss function. Model parameters of the machine learning model may be modified to reduce the differences or to reduce the loss values to a (defined) minimum.

The machine learning model may, for example, be an artificial neural network or include such a network.

An artificial neural network comprises at least three layers of processing elements: a first layer with input neurons (nodes), an nth layer with at least one output neuron (nodes) and n-2 inner layers, where n is a natural number and greater than 2.

The input neurons serve to receive the radiological images. There is usually one input neuron for each picture element/volume element of a radiological image. There may be additional input neurons for additional input values (e.g. information about the examination region, about the examination object and/or about conditions which prevailed when generating the radiological image).

The output neurons can serve to output a segmented representation.

The processing elements of the layers between the input neurons and the output neurons are connected to one another in a predetermined pattern with predetermined connection weights.

Preferably, the artificial neural network is a so-called convolutional neural network (CNN) or comprises such a network.

A CNN normally consists essentially of an alternately repeating array of filters (convolutional layer) and aggregation layers (pooling layer) terminating in one or more layers of “normal” fully connected neurons (dense/fully connected layer).

The training of the neural network may, for example, be carried out by means of a back propagation method. The goal for the network is to predict the segmented radiological image as reliably as possible. The quality of prediction may be described by a loss function. The goal is to minimize the loss function. In the case of the back propagation method, an artificial neural network is taught by the change of the connection weights.

In the trained state, the connection weights between the processing elements contain information regarding the segmentation of the pancreas that can be used to generate (predict) on the basis of a new unsegmented radiological image a segmented radiological image. The term “new” means that the corresponding radiological image was not used during the training.

A cross-validation method may be used to divide the data into training data sets and validation data sets. The training data set is used in the back propagation training of network weights. The validation data set is used to check the accuracy of prediction with which the trained network can be applied to unknown data.

The artificial neural network may have an autoencoder architecture, for example the artificial neural network may have an architecture such as the U-Net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network may be a generative adversarial network (GAN) (see for example M.-Y. Liu et al.: Generative Adversarial Networks for Image and Video Synthesis: Algorithms and Applications, arXiv: 2008.02793; J. Henry et al.: Pix2Pix GAN for Image-to-Image Translation, DOI: 10.13140/RG.2.2.32286.66887).

The artificial neural network may be a transformer network (see for example D. Karimi et al.: Convolution-Free Medical Image Segmentation using Transformers, arXiv: 2102.13645 [eess.IV]).

The machine learning model serving for segmentation of the pancreas from the at least one radiological image is also referred to in this description as the first machine learning model.

Another way of segmenting the pancreas from the at least one radiological image is atlas-based segmentation. Atlas-based segmentation uses prior knowledge about the object to be located in the form of a model (the atlas). The prior knowledge may include knowledge about the shape, orientation, continuity, elasticity and/or smoothness of the object to be segmented.

An atlas-based method can generate a two-or three-dimensional model of the pancreas. In such a model, the atlas, the shape of the object to be segmented is predefined. The atlas may be, for example, a (manually) segmented radiological image of an average pancreas. Ways of generating atlases are mentioned later in the description.

In atlas-based segmentation, the picture elements/volume elements that are attributable to the pancreas (that represent part of the pancreas) are known for the atlas. In atlas-based segmentation, the atlas is subjected to one or more transformations for optimum matching thereof with the at least one radiological image. The finding of the transformation(s) that achieve(s) optimum matching of the atlas with the at least one radiological image is also referred to as registration. Once registration has been completed, the matching picture elements/volume elements that represent the pancreas in the case of the atlas can also be labeled as the picture elements/volume elements attributable to the pancreas in the case of the at least one radiological image.

A distinction can be made between three transformations in registration: a rigid transformation, an affine transformation and an elastic transformation. A rigid transformation allows translations and rotations of the atlas and thus has six degrees of freedom in three-dimensional space. An affine transformation allows not only translations and rotations, but also scaling and shear mapping. It has twelve degrees of freedom in three-dimensional space. The term “elastic transformation” usually covers various transformation models that can have different degrees of freedom. An elastic transformation model comprises, for example, a grid which can be placed over the at least one radiological image and in which the individual grid points are movable, such that the at least one radiological image can be skewed accordingly. This allows the atlas to be adjusted not only in relation to size and orientation, but also in relation to shape.

Since the pancreas can vary in shape between examination objects, meaning that rotation, translation, scaling and/or shear mapping are often insufficient for optimum mapping of the atlas onto the at least one radiological image, preference is given to using an elastic transformation model.

Examples of elastic transformation models are BSpline registration, finite element registration, Demons registration, level set motion registration, and thin plate spline registration.

Registration is usually an iterative method in which various transformations are performed one after the other, and a check is carried out for each transformed atlas to determine how good the match is between the transformed atlas and the at least one radiological image. In order to check how well the atlas fits onto the at least one radiological image, what can be determined is a similarity measure, which is based on brightness values of the picture elements/volume elements, on their spatial distribution, on a histogram evaluation and/or on other/further features. Examples of functions for calculation of a similarity measure are sum of squared differences, and mutual information.

The performance of transformations and the finding of the transformation(s) that ensure(s) an optimum match is usually not done aimlessly; instead, use can be made of known optimization methods such as gradient-based methods (e.g. gradient descent, Newton's method) or non-gradient-based methods (e.g. Powell's method, downhill simplex method, best neighbor method, golden ratio).

An atlas may be, for example, a segmented radiological image of an average pancreas, which is obtained, for example, by averaging a multiplicity of radiological images of a multiplicity of different examination objects.

Such an atlas may be generated, for example, by registration and superimposition of multiple radiological images with a reference image and averaging. The reference image may be a radiological image of a pancreas having a shape, size and/or orientation that is frequently observed (average pancreas).

Before the radiological images are matched with one another, they are usually segmented (manually, e.g. by a radiologist).

The radiological images may be registered with the reference image one after another and each individual registration may be followed by averaging of the radiological image with the reference image to generate a new reference image with which the next radiological image is then registered and fused. However, it is also conceivable that multiple radiological images are first registered with the reference image before the radiological images are averaged together with the reference image to form a new reference image (or the final atlas). When averaging two radiological images, the gray values of the picture elements/volume elements of the individual images may for example be divided by two in each case in order to then add up the gray values of the two images that were divided by two. When averaging n radiological images, the gray values of the picture elements/volume elements of the individual images may be divided by the number n in each case in order to then add up the gray values of all the images that were divided by n, where n is an integer.

Different atlases may be used for men and women. Likewise, different atlases may be used for children and adults. Different atlases may be used, for example, for different age groups.

Further details on atlas-based segmentation can be found in the prior art (see for example: H. Park et al.: Construction of an Abdominal Probabilistic Atlas and its application in Segmentation, IEEE Transactions on medical imaging, 2003, Vol. 22, 483-492; P. Slagmolen et al.: Atlas based liver segmentation using nonrigid registration with a B-spline transformation model, MICCAI 2007, 197-206; O. Commowick et al.: Atlas-Based Delineation of Lymph Node Levels in Head and Neck Computed Tomography Images, In Radiotherapy Oncology, 2008, 87(2), 281-289; A. Shimizu et al.: Segmentation of multiple organs in non-contrast 3D abdominal CT images, International Journal of Computer Assisted Radiology and Surgery, 2007, Vol. 2, No. 3-4, 35-142).

Another way of segmenting the pancreas from the at least one radiological image is to use a deformable model. The main difference between atlas registration and a deformable model lies in the formulation of the problem. Deformable models are usually implemented as physical bodies having a defined elasticity and a force that attempts to maintain the shape of the bodies, whereas the at least one radiological image to be segmented is usually represented as a potential field having a force that attempts to stretch and/or compress the model for fitting thereof. The optimum fit may be found as a solution with minimum energy, in which both forces are in equilibrium.

A deformable model of a pancreas may be obtained from a multiplicity of segmented radiological images of the pancreas of a multiplicity of examination objects. What may be obtained from the multiplicity of segmented radiological images is an average pancreas. Likewise, the variability of the pancreas in relation to size and/or shape, which can be observed in the multiplicity of segmented radiological images, may be obtained from the radiological images. This variability can define the range of permissible deformations, i.e., deformations that are not observed in real life can be prohibited or be associated with a comparatively high force that must be applied in order to realize the corresponding deformation. For deformations that occur frequently, the force required for realization thereof is comparatively low.

Further details on segmentation using a deformable model can be found in the prior art (see for example: S.F.F. Gibson et al.: A Survey of Deformable Modeling in Computer Graphics, Tech. Rep., 1997; T. McInerney et al.: Deformable models in medical image analysis: a survey, Medical Image Analysis, 1996, Vol. 1, No. 2, 91-108; C. Xu et al.: Image Segmentation Using Deformable Models, Handbook of Medical Imaging, SPIE Press, 2000, Vol. 2., 447-514; A. Konno et al.: A Hepato-Biliary-Pancreatic Deformable Model for a Simulation-Based Laparoscopic Surgery Navigation, 2020 IEEE/SICE International Symposium on System Integration (SII), 2020, 39-44, doi: 10.1109/SII46433.2020.9025967; O. Ecabert et al.: Automatic Model-Based Segmentation of the Heart in CT Images, IEEE Transactions on Medical Imaging, Vol. 27, No. 9, 2008, 1189-1201).

In a further step, parts of the pancreas may be segmented from the at least one (preferably segmented) radiological image. The parts are preferably parts of the pancreas as present in any (healthy) examination object.

The pancreas is frequently divided into three parts: the head of the pancreas, the body of the pancreas and the tail of the pancreas, also referred to as head, body and tail for short (see for example FIG. 4 in: T. Ohtsuka et al.: Modern Japanese Approach to Pancreatic Cancer, Pancreatic Cancer, 2016, Springer, https://doi.org/10.1007/978-1-4939-6631-8_49-2).

In some publications, a division into five parts is made: hook (Processus uncinatus pancreatis), head, neck, body and tail (see for example: K.M. Ramonell et al.: Pancreas, Surgical Anatomy and Technique, Springer, 2021, https://doi.org/10.1007/978-3-030-51313-9_9).

The main pancreatic duct can be considered to be a further part of the pancreas.

Preferably, the segmentation of parts of the pancreas in the at least one radiological image comprises the segmentation of one or more of the following parts: hook, head, neck, body, tail, main pancreatic duct.

Preferably, at least the main pancreatic duct is segmented from the at least one radiological image.

As described for the segmentation of the pancreas, one or more of the aforementioned parts may be segmented from the at least one radiological image with the aid of a machine learning model. The machine learning model may be trained on the basis of training data in which one or more parts of the pancreas has/have been segmented (e.g. manually by a radiologist). The machine learning model may be trained to apply the segmentation, which was performed manually for example with the training data, to new radiological images. It is conceivable that different machine learning models exist for different parts to be segmented, or that one machine learning model is trained to segment a plurality of different parts from radiological images. The machine learning model used for segmentation of the pancreas from the at least one radiological image may have been trained, for example, to simultaneously segment the pancreas as a whole and one or more parts of the pancreas from the at least one radiological image. Furthermore, the machine learning model may have been trained to segment one or more parts of the pancreas from the at least one radiological image originally received.

Furthermore, one or more parts of the pancreas may be segmented by an atlas-based segmentation method and/or with the aid of a deformable model from the at least one radiological image.

### Segmentation and/or Identification of the Main Pancreatic Duct

A goal of identification and/or characterization of cystic lesions of the pancreas may be to identify and/or characterize cysts in the main pancreatic duct. In order to detect lesions in the main pancreatic duct, in a first step the main pancreatic duct may be segmented in the at least one radiological image, as described above.

In a preferred embodiment, a centerline is determined for the main pancreatic duct segmented from the at least one radiological image. A centerline is an imaginary line running along the main pancreatic duct. A centerline can be understood to mean a line onto which the main pancreatic duct can be shrunk. A centerline shows the course of the main pancreatic duct in the form of a one-dimensional curve from a beginning to an end, the information concerning the extent of the main pancreatic duct into the other two dimensions having been reduced to zero.

There are various methods for determining a centerline (see for example: M. Lenga et al.: Deep Learning Based Rib Centerline Extraction and Labeling, https://doi.org/10.1007/978-3-030-11166-3_9; A. Sironiet et al.: Multiscale Centerline Detection, IEEE Transactions on Pattern Analysis and Machine Intelligence, 2015, 38. 1-1. 10.1109/TPAMI.2015.2462363; U.S. Pat. No. 8,229,186B2).

The present invention is not limited to a specific method.

The abovementioned methods for determining a centerline require that the main pancreatic duct can be segmented from the at least one radiological image. However, in some radiological images, the main pancreatic duct cannot be detected or only part of it can be detected, meaning that segmentation, in particular by means of an atlas-based segmentation method or by means of a segmentation method based on a deformable model, can cause difficulties.

Therefore, in a preferred embodiment, a centerline is predicted. To this end, in a first step the pancreas is segmented from the at least one radiological image, as described in this description. The at least one radiological image is a three-dimensional image of the pancreas or a stack of two-dimensional images of the pancreas from which a three-dimensional representation can be constructed. Methods for converting three-dimensional representations into a stack of two-dimensional representations and vice versa are described in the prior art (see for example: Aharchi M., Ait Kbir M.: A Review on 3D Reconstruction Techniques from 2D Images, DOI: 10.1007/978-3-030-37629-1_37; Ehlke, M.: 3D-Rekonstruktion anatomischer Strukturen aus 2D-Röntgenaufnahmen, DOI: 10.14279/depositonce-11553; https://neuro.debian.net/pkgs/nifti2dicom.html).

Thus the segmented pancreas is preferably a three-dimensional representation. Optionally, one or more parts of the pancreas may have been segmented. From the at least one radiological image that has been segmented, the volume elements (voxels) outwardly delimiting the pancreas can be determined. In other words: from the at least one radiological image that has been segmented, a surface of the pancreas can be derived. Optionally, such a surface includes information concerning the part of the pancreas to which individual volume elements belong, i.e. the part of the pancreas that they represent. In a further step, the surface representation of the pancreas may be fed to a machine learning model. The machine learning model may have been trained on the basis of training data to predict on the basis of the surface representation a centerline. The training data may comprise segmented radiological images of the pancreas of a multiplicity of examination objects, and in the segmented radiological images both the surface of the pancreas has been labeled (segmented) and a centerline for the main pancreatic duct has been labeled. Thus the machine learning model may have been trained to predict from a surface representation of a pancreas a centerline. The machine learning model may be trained by using segmented radiological images of a modality that is the same or different with respect to the modality when using the trained machine learning model for prediction. It is thus possible, for example, to obtain segmented radiological images from MRI images for the training of the machine learning model, to train the model on the basis of the segmented MRI images, and to use the trained model for prediction of a centerline on the basis of at least one CT image. It is also possible to obtain segmented radiological images from CT images for the training of the machine learning model, to train the model on the basis of the segmented CT images, and to use the trained model for prediction of a centerline on the basis of at least one MRI image.

### Identification and/or Characterization of Dilatations in the Main Pancreatic Duct

In a further step, dilatations may be identified in the main pancreatic duct. Such dilatations may indicate the presence of a cyst, a lesion or a cystic lesion in the corresponding region (see for example I. Karoumpalisa, D. K. Christodoulou: Cystic lesions of the pancreas, Ann Gastroenterol. 2016 April-Jun; 29(2): 155-161).

Different methods may be used to identify dilatations along the centerline of the main pancreatic duct. The present invention is not limited to a specific method.

In one embodiment, a plane is moved along the centerline of the main pancreatic duct and the regions of intersection of the plane with the segmented main pancreatic duct are determined. Preferably, the plane at each point along the centerline is perpendicular to the centerline. In other words: a tangent is determined at preferably each point of the centerline and a plane is spread out around the point, with the respective tangent representing the surface normal (also called normal vector) in relation to the respective plane. For each point, the region of intersection of the respective plane with the segmented main pancreatic duct is determined.

An increase in the region of intersection (an increase in the intersection area) may indicate the presence of a cyst or a lesion or a cystic lesion in the main pancreatic duct. For each point of the centerline, the size of the determined region of intersection may be compared with a predefined threshold. If the size of the determined region of intersection is above a defined threshold range, this may indicate a cystic lesion. Regions of intersection of planes of adjacent points of the centerline with the segmented main pancreatic duct may be combined to form a volume model of the main pancreatic duct, from which the volume of a dilatation and the extents into the three spatial directions can be determined.

In a further embodiment, a machine learning model, which may have been configured as a regression model or classification model, may be used to detect and/or classify dilatations. For example, a regression model may be trained to determine the mean (e.g. arithmetically averaged) radius or the mean (e.g. arithmetically averaged) diameter of the main pancreatic duct for points along the centerline. It is also possible to train the model to determine the maximum radius or the maximum diameter of the main pancreatic duct. The regression model may also be trained to determine the size of the area of intersection of the main pancreatic duct with a plane perpendicular to the tangent of the centerline at a series of points of the centerline. A classification model may be trained to divide sectional images running through the main pancreatic duct into one of at least two classes, where one class contains for example images of normal (i.e. healthy) main pancreatic ducts and at least one further class includes images of dilated main pancreatic ducts, where the respective class can indicate the cause of the dilatation. In other words: the classification model may be trained to classify dilatations of the main pancreatic duct, where the class can indicate the dilatation type and/or the cause of the dilatation.

The regression and/or classification model may be trained on the basis of sectional images of the main pancreatic duct, which are for example moved along the main pancreatic duct in the manner of a so-called “sliding window” or “rolling window” (see for example: A. Helwan et al.: Sliding Window Based Machine Learning System for the Left Ventricle Localization in MR Cardiac Images, DOI: 10.1155/2017/3048181).

Dilatations of the main pancreatic duct may be characterized after they have been detected. The characterization may be, for example, a classification in which each detected dilatation is assigned to one of multiple classes, where the respective class can indicate what the dilatation type is and/or what the cause(s) of the dilatation may be.

A dilated main pancreatic duct may be caused by a benign or malignant disease or by solid or cystic pancreatic tumors. The main pancreatic duct may also be dilated owing to chronic pancreatitis or as a result of aging or normal physiological processes.

In one embodiment, each detected dilatation of the main pancreatic duct is assigned to one of at least two classes, where one class contains dilatations attributable to intraductal papillary mucinous neoplasms.

The classification of dilatations may be done, for example, with the aid of a machine learning model which has been trained on the basis of training data to detect at least one dilatation type (e.g. intraductal papillary mucinous neoplasms) and to assign at least one radiological image showing such a dilatation type to a class containing this dilatation type.

In a further embodiment, a model based on machine learning is used for direct segmentation and classification, in one or more radiological images, of regions of the centerline of the main pancreatic duct that may have been dilated. Preferably, the model operates on a region of interest that includes the main pancreatic duct. Said region of interest may be determined automatically; for example, it may be a bounding box which encloses the segmented pancreas or the segmented main pancreatic duct. Alternatively, said region of interest may be defined by a user, for example by a user drawing a bounding box around the region of interest by means of a graphical user interface. The machine learning model may have been configured to segment one or more radiological images or one or more regions of interest in one or more radiological images and, at the same time, to mark dilatations of the main pancreatic duct. Such a machine learning model may be based, for example, on an autoencoder which receives input data (e.g. one or more radiological images which are segmented or unsegmented and/or provided with one or more bounding boxes), generates a compressed representation of the input data and, on the basis of the compressed representation, generates and outputs one or more segmented radiological images in which dilatations in the main pancreatic duct have been highlighted and/or marked. An example of the implementation of such a model is the so-called U-Net (see for example: O. Ronneberger et al.: U-Net: Convolutional Networks for Biomedical Image Segmentation, https://doi.org/10.48550/arXiv.1505.04597). Furthermore, the machine learning model may have been trained to classify (segmented) dilatations.

The use of a model-based approach for identification and/or characterization of dilatations of the main pancreatic duct, as described above for the segmentation of the pancreas, may be used too.

### Identification and/or Characterization and/or Localization of Lesions in the Pancreas

Besides cystic lesions within the main pancreatic duct, cystic lesions in other regions of the pancreas may also be detected and characterized.

In a further step, one or more cysts, lesions or cystic lesions are identified in the segmented and identified parts of the pancreas. Different methods may be used to identify possible cysts in the identified parts of the pancreas. The present invention is not limited to a specific method.

In one embodiment, possible cysts, lesions or cystic lesions in the segmented regions of the pancreas may be classified according to their type and/or cause. The method provided in the present invention allows the identification and/or classification of various types of cysts and/or lesions, including cystic lesions. For details about classification of the types and causes of lesions in the pancreas, see for example “Ann Gastroenterol. 2016 April-June; 29 (2): 155-161, doi: 10.20524/aog.2016.0007” and the references cited therein. The classification of the lesions may be done once again with a neural network or a statistical shape model, for example.

In a further embodiment, cysts, lesions or cystic lesions of the segmented and identified parts of the pancreas may be identified using a “bounding box detector” or a “minimum bounding rectangle”. This may be done either two-dimensionally with the aid of slices or three-dimensionally, i.e. volumetrically. In a preferred embodiment, the two-dimensional identification of lesions may be achieved by the YOLO object detection approach. This method uses a neural network for prediction of bounding boxes. In a further preferred embodiment, the three-dimensional identification of lesions may be achieved using a volumetric bounding box detector variant.

In a further embodiment, possible lesions in the pancreas may be classified by determining the corresponding center of gravity (COG) of each cyst or lesion with a landmark detector or keypoint detector.

### Determination of One or More Measurement Values

The identification and classification of possible cysts, lesions or cystic lesions in the pancreas and/or along the centerline of the pancreas provides important information about a number of different parameters, including, but not limited to, the number of lesions in the pancreas, their location within the tissue, including their location in and within a pancreatic subregion (e.g. coordinates, size), their morphology, i.e. their maximum and/or mean (e.g. arithmetically averaged) diameter and/or a Feret diameter of the lesions (e.g. in mm), their volume (e.g. in mm³), their sphericity (see e.g. http://dx.doi.org/10.1680/jgeot.16.P.207) and/or their interactions (e.g. whether the lesions intersect, overlap, or are related to each other in some way).

The aforementioned measurement values and other/further measurement values may be obtained using known image analysis methods (see for example: D. Luo: Pattern Recognition and Image Processing, Elsevier Science, 1998, ISBN: 9780857099761). There are also commercially available and freely available software which may be used to determine the aforementioned features and further features. One example is the software BioVoxxel (https://www.biovoxxel.de/).

### Outputting of at Least One Representation of the Pancreas

In a further step, at least one representation of the pancreas is output, the one or more identified and characterized cysts, lesions or cystic lesions having been labeled in the at least one representation.

In one embodiment, a representation of the pancreas is provided, said representation comprising the presentation of the information obtained in earlier steps and/or embodiments concerning the identification and classification of possible cysts, lesions or cystic lesions in one or more segments of the pancreas or in the centerline of the pancreas. This representation enables the recipient of the information to make a qualified assessment and to diagnose possible states which are a threat to the health of a subject. The recipient of the information is preferably a person with medical training, particularly preferably a person with advanced medical training, particularly preferably a technician, particularly preferably a physician. The information display may include features that enable the recipient to interact with the information or to alter the information display, i.e. by changing for example the view, scale, perspective or other appropriate parameters of the images. In a preferred embodiment, the recipient of the information may use their medical knowledge to alter the displayed information, for example to manually correct parameters such as the position of the centerline of the pancreas or the edges of a possible lesion, cyst or cystic lesion.

In a preferred embodiment, the computer system according to the invention executes steps described in the present description one after another and outputs, after each individual step or group of steps, a representation of the pancreas or of a body region comprising the entire pancreas or part of the pancreas to a user, so that the user can check the result of individual steps or groups of steps and, if necessary, take corrective action.

Thus the computer system according to the invention may have been configured for example to output the segmented pancreas. The user can check whether the segmentation is correct, i.e. whether all the regions in the at least one radiological image that were assigned to the pancreas by the computer system were correctly assigned. In the event of the computer system according to the invention failing to correctly assign a region, the computer system may have been configured to accept an input from the user, for example by means of a computer mouse, that provides details of the correspondingly incorrectly assigned region. The computer system according to the invention may have been configured to make an appropriate correction, i.e. to mark the region as specified by the user as belonging to the pancreas or not belonging to the pancreas, before the computer system according to the invention continues with subsequent steps.

This also applies analogously to other segmentations, for example the segmentation of the constituents of the pancreas, in particular the main pancreatic duct, the dilatations in the main pancreatic duct and/or identified cystic lesions.

1. A computer-implemented method for identifying and characterizing cystic lesions of the pancreas, comprising:
receiving at least one radiological image, the at least one radiological image showing a pancreas of an examination object,
segmenting the pancreas from the at least one radiological image,
segmenting parts of the pancreas from the at least one radiological image,
identifying one or more cysts in the identified parts of the pancreas,
classifying the one or more cysts,
determining one or more measurement values for the one or more cysts,
displaying at least one representation of the pancreas, the one or more cysts having been labeled in the at least one representation.

2. A system comprising a processor, memory, and a program stored in the memory for execution by the processor, the program including instructions that when executed by the processor cause the system to:
load at least one radiological image into the memory, the at least one radiological image showing a pancreas of an examination object,
segment the pancreas from the at least one radiological image,
segment parts of the pancreas in the at least one radiological image,
identify one or more cysts in the identified parts of the pancreas,
classify the one or more cysts,
determine one or more measurement values for the one or more cysts,
display at least one representation of the pancreas, the one or more cysts having been labeled in the at least one representation.

3. A computer-readable medium storing instructions, wherein the instructions are executable by a system comprising a processor to cause the system to:
load at least one radiological image into a memory, the at least one radiological image showing a pancreas of an examination object,
segment the pancreas from the at least one radiological image,
segment parts of the pancreas from the at least one radiological image,
identify one or more cysts in the identified parts of the pancreas,
classify the one or more cysts,
determine one or more measurement values for the one or more cysts,
display at least one representation of the pancreas, the one or more cysts having been labeled in the at least one representation.

4. The method of claim 1, wherein the segmentation of the pancreas from the at least one radiological image comprises at least one of an identification and segmentation of a main pancreatic duct and a determination of a centerline in the main pancreatic duct.

5. The method of claim 1, wherein the segmentation of parts of the pancreas from the at least one radiological image comprises at least one of an identification and segmentation of a main pancreatic duct and a determination of a centerline in the main pancreatic duct.

6. The system of claim 2, wherein the segmentation of the pancreas from the at least one radiological comprises at least one of an identification and segmentation of a main pancreatic duct and a determination of a centerline in the main pancreatic duct.

7. The system of claim 2, wherein the segmentation of parts of the pancreas from the at least one radiological image comprises at least one of an identification and segmentation of a main pancreatic duct and a determination of a centerline in the main pancreatic duct.

8. The computer-readable medium of claim 3, wherein the segmentation of the pancreas from the at least one radiological comprises at least one of an identification and segmentation of a main pancreatic duct and a determination of a centerline in the main pancreatic duct.

9. The computer-readable medium of claim 3, wherein the segmentation of parts of the pancreas from the at least one radiological image comprises at least one of an identification and segmentation of a main pancreatic duct and a determination of a centerline in the main pancreatic duct.
